(19) United States
(12) Patent Application Publication
(10) Pub. No.: US 2002/0197685 A1
(43) Pub. Date: Dec. 26, 2002

(54) Amplification of heterogeneous full-length mRNA

(76) Inventor: Ming Zhou, San Diego, CA (US)

Correspondence Address: Ming Zhou, 13378 RUSSET LEAF LANE, San Diego, CA, 92129, US

(21) Appl. No.: 10/174,739

(22) Filed: Jun. 19, 2002

Related U.S. Application Data
(60) Provisional application No. 60/299,413, filed on Jun. 20, 2001.

(51) Int. Cl. C12P 19/34; C12Q 1/68

(52) U.S. Cl. 435/91.2; 435/6

(57) ABSTRACT

An in vitro method for unbiased amplification of heterogeneous full length mRNA is described. The amplified full-length mRNA can be used to amplify the protein content of a given type of cells/tissues when coupled with in vitro translation system. This method finds applications in biology and medicine, including analysis of gene function, differential gene expression, protein discovery, cellular and clinical diagnostics and drug screening.

### CROSS-REFERENCE TO RELATED APPLICATION

This application claims the benefit of the priority date of provisional application Ser. No. 60/299,413 filed Jun. 20, 2001, the contents of which are incorporated herein by reference in their entirety

### FEDERALLY SPONSORED RESEARCH OR DEVELOPMENT

Not applicable

### FIELD OF THE INVENTION

The present invention relates to a method for making full-length mRNA

### BACKGROUND OF THE INVENTION

Characterization of gene expression finds applications in a variety of disciplines, such as in analysis of differential expression between different tissue types, different stages of cellular growth or between normal and disease states. There are two fundamental approaches to gene expression analysis The first one is the DNA microarray technology, which has been widely used to characterize gene expression at mRNA level However, mRNA study are often complicated by one or more of the following factors cell heterogeneity, material paucity, and detection limitation for low-abundance mRNA The second approach is termed proteomics for global analysis of proteins in a given type of cells/tissues. Since proteins are the main functional output and the cellular mRNA levels do not necessarily correlate with the expression levels of gene products, proteomics research has attracted much attention in the post-genome era. Proteomic analysis is most commonly accomplished by a combination of sophisticated techniques including two-dimensional (2D) gel electrophoresis (for separation, visualization and quantification), mass spectrometry (for identification), and bioinformatics (for function analysis) This is a tedious, time-consuming process. And the quantity of sample is often limited, making sample preparation the most challenging step in proteomic analysis. Furthermore, proteins that are of biological importance, such as enzymes and receptors, are often present as rare cellular components, making the detection of such proteins even more difficult.

In the past two decades, there have been great achievements in biomedical research with regard to enhancing the detection sensitivity of biomolecules The most common technique is known as PCR (Polymerase Chain Reaction)-based cDNA amplification (U.S. Pat. No. 5,643,766 to Scheele, et al. (1997) and U.S. Pat. No. 6,110,711 to Serafini et al.(2000)) To utilize this technology, one first makes cDNA from RNA using reverse transcriptase, followed by addition of a homopolymer tail (such as a tandem cytosine) with terminal transferase, or an arbitrary primer through DNA ligation to the 3′-end of the first strand cDNA The amplification process utilizes the added sequence and the poly-A tail of second strand cDNA as priming binding sites. PCR technology, however, suffers from a serious drawback. It is well known that PCR works best when small regions of a few hundred nucleotides are being amplified. When heterogeneous cDNAs are used as templates, amplification will be a disproportionate process such that longer cDNAs are not amplified at the same rate as shorter cDNAs. Therefore, even a small difference in efficiency will result in a biased amplified cDNA population. In addition, the error rate of the enzyme most commonly used for PCR (such as Taq polymerase) is high, so it is certain that most PCR-amplified cDNAs will contain several erroneous bases. These technological problems currently limit the overall usefulness of PCR in the study of gene expression.

Another method developed to address at least some of the above problems associated with mRNA detection was known as antisense RNA (aRNA) amplification (U.S. Pat. No. 5,514,545 to Eberwine (1996), and U.S. Pat. No. 5,932,451 to Wang et. al. (1999)) In this method the first strand cDNA is prepared from mRNA using an oligo dT primer that comprises an RNA polymerase promoter region 5′ of the oligo dT region The first strand cDNA is then converted to ds cDNA To produce aRNA, the ds cDNA is employed for in vitro transcription with the appropriate RNA polymerase However, one application limitation with antisense RNA amplification is that the resulting product aRNAs, unlike cellular mRNAs, can't be used as templates for in vitro translation.

Accordingly, it has become a real challenge and a necessity in gene expression profiling, both at the transcription level (mRNA) and the translation level (proteins), to develop a robust system for in vitro amplification of the complete set of mRNA in a given type of cells/tissues.

### SUMMARY OF THE INVENTION

This invention relates to a novel method for unbiased amplification of heterogeneous, cellular full-length mRNA for gene expression profiling, meaning to characterize both mRNA (transcription) and protein (translation) for any given type of cells/tissues In addition, this invention relates to the emerging field of proteomics, which involves the systemic identification and characterization of proteins that are present in biological samples so that their role in health and disease can be determined. Such information is valuable for diagnosis, prognosis, or monitoring response to therapy, and in elucidating disease mechanisms and identifying therapeutic targets for the prevention and treatment of disease

### BRIEF DESCRIPTION OF THE FIGURE

Referring particularly to the figure for the purpose of illustration only and not limitation, there is illustrated:

FIG. 1 is a scheme showing each step of the method for unbiased amplification of full-length mRNA The method comprises several steps: Dephosphorylation of RNA (total or mRNA); Removal of the 5′ end cap structure (m₇Gppp) from the full-length mRNA, Addition of a synthetic RNA adapter containing an RNA polymerase site to 5′ end of the decapped mRNA, Synthesis of ss cDNA and ds cDNA; and Production of amplified mRNA through in vitro transcription.

FIG. 2 is a scheme showing the steps of generating an array of individual proteins. These steps include Making gene-specific expressed sequence tags (EST), Immobilizing the tags to predetermined, addressable locations in a matrix to form an array; Carrying out an in vitro unbiased amplification of heterogeneous full length mRNA, Applying the amplified mRNA molecules to the array, followed by incubation to allow coupling of mRNA to their complementary capture tags, Removing non-complementary mRNA, Carrying out synthesis of protein in situ by in vitro translation of captured mRNA in the array. Here, a Stands for full-length mRNA molecules containing a sequence complementary to the capture tag; b Stands for truncated mRNA molecules that contains a sequence complementary to the capture tag; c Stands for mRNA not containing a sequence complementary to the capture tag, d Stands for other RNA or non-RNA molecules

### DESCRIPTION OF THE INVENTION

The object of the present invention is to prepare the unbiased amplification of full-length mRNA from any given type of cells/tissues so as to facilitate gene expression profiling of these cells/tissues In principle, it consists of several steps described in FIG

A RNAs (total or mRNA) are treated with calf intestinal phosphatase (CIP) to remove the 5′-phosphates from truncated mRNAs and non-mRNAs. CIP has no effect on the full-length mRNAs, which contain the cap structure

B. Use tobacco acid pyrophosphatase (TAP) to remove the cap structure (Gppp.triphosphate) from the full-length mRNAs, leaving a 5′-monophosphate for subsequent ligation reaction.

C Ligation reaction is accomplished by T4 RNA ligase between the decapped mRNAs and a synthetic RNA adapter containing an RNA polymerase site (such as the T7 RNA Polymerase binding site (5′ AAA CGA CGG CCA GTG AAT TGT AAT ACG ACT CAC TAT AGG GCG 3′).

D. Synthesis of first-strand cDNAs with reverse transcriptase (such as SuperScript II, Life Technologies) and an anchor oligo-dT, in which immediately 3′ of the oligo dT region is either a “G,” “C” or “A” such that the primer has the configuration of 3′-XTTT 5′, where X is either “G,” “C” or “A”.

E. RNase H digestion (removal of the template mRNAs from the RNA/DNA hybrids)

F Synthesis of double-strand cDNAs using DNA polymerase (such as Pfu) and a DNA oligonucleotide primer complementary to the RNA adapter, which has a capturable moiety (e.g biotin) at its 5′ terminus.

G The full-length, double-strand cDNAs is captured on a solid phase through specific binding interaction between the first moiety (e g biotin) at the 5′ terminus of the primer and the second moiety (e.g streptavidin) associated with a solid support. Specific solid phases of interest include polystyrene pegs, sheets, beads, magnetic beads, and the like.

H The captured cDNAs will serve as templates in an in vitro transcription system (such as MEGAscript in vitro translation kit, Ambion) with the appropriate RNA polymerase, (e.g T7 polymerase) to make “amplified full-length mRNAs”. The amplified material will be similar in size distribution to the parental mRNAs and will show sequence heterogeneity as well.

### DESCRIPTION OF THE SPECIFIC EMBODIMENT

Traditionally, genome-wide analysis for protein function is carried out with cDNA expression libraries. Most frequently, the libraries are prepared in phage vectors and the expressed proteins immobilized on a membrane by a plaque lift procedure Although this approach has some applications (Young R. A. and Davis R. W. Science 222, 778, (1983), Sparks A. B. et. al Nature Biotechnol. 14, 741, (1996); Fukunaga R and Hunter T. EMBO J. 16, 1921, (1997), Tanaka H., Mol. Pharmacol. 55, 356, (1999)), it has many limitations Most noticeably, the majority of the clones in the library do not encode proteins in the correct reading frame, and most proteins are not full-length.

More recently, advances in protein identification using mass spectrometry have facilitated protein profiling in biological samples The most widespread strategy with this technology employs two-dimensional polyacrylamide gel electrophoresis (2D PAGE) followed by enzymatic degradation of isolated protein spots, peptide mapping, and bioinformatics searches Using this method, several thousand proteins can be resolved in a gel and their expression quantified. However, many proteins possessing important cellular functions are not easily analyzed using this strategy. These include membrane proteins, low copy number proteins, highly basic proteins, and very large (>150 kDa) or small (<10 kDa) proteins

Complementary to the above technology, protein microarrays, or protein chips, are now being developed and modified to a high-throughput screening format. The protein microarrays make it possible to develop a rapid global analysis of the entire proteome In one example of such approach, individual proteins are spotted onto chemically derivatized glass slides using a high-precision robot, which was originally designed to manufacture complementary DNA (cDNA) microarrays (MacBeath G and Schreiber S. L. Science 289, 1760-1763, (2000)) The proteins attached covalently to the slide surface yet retained their ability to interact specifically with other proteins, or with small molecules, in solution. The functions of the proteins on the slide can be studied simultaneously In another example, protein chips were prepared by nano-spotting of recombinant scFv antibody fragments onto micro-engineered silicon chips (Borrebaeck C. A K et al, BioTechniques 30, 1126-1132 (2001)) Such protein chips allow the determination of single or multiple antigen-antibody interactions Although these approaches have been shown to have great potential in rapid elucidation of protein functions, they suffer a serious limitation as acknowledged by the authors—they all rely on the availability of isolated proteins and cDNA constructs Currently there is no convenient technique to produce a comprehensive set of individual proteins that are expressed in a biological system

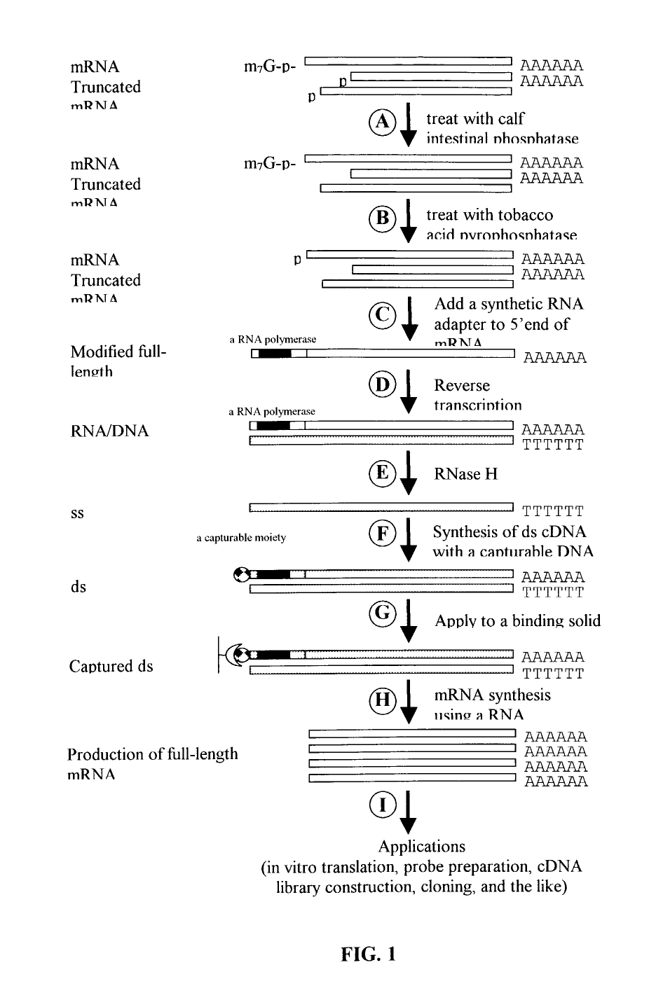

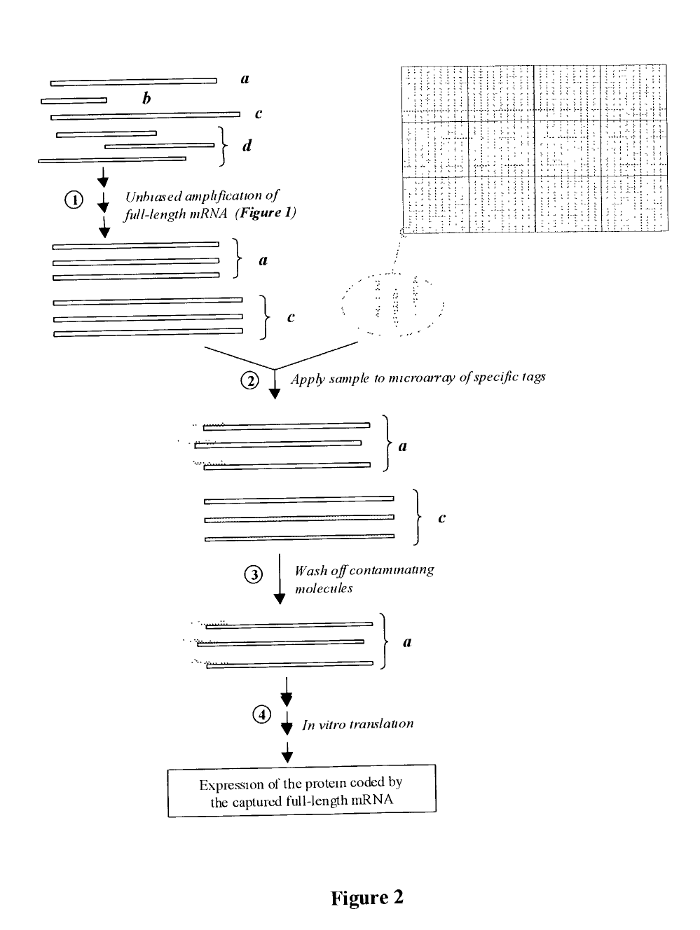

The present invention provide a convenient means of preparing microarrays of individual proteins in any given type of cells or tissues so as to facilitate the structure characterization and function determination of the proteins. In principle, the method consists of the following steps as described in FIG. 2 (i) specific capture tags are designed for every protein based on its corresponding expressed sequence tags (EST) sequence (ii) The capture tags are synthesized, and are immobilized to predefined locations in a matrix to form an array of capture tags in a multiple-well formatted plate so that each spot of the array contains only one specific type of capture tags (iii) Carry out unbiased amplification of full length mRNA according to the steps described in FIG. 1. (iv) The amplified mRNA molecules are applied to the microarray, followed by incubation to allow coupling of mRNA to their complementary capture tags. (v) mRNA molecules that do not contain a sequence complementary to the capture tags are removed after washing, while mRNA molecules containing a sequence complementary to the capture tags are retained. (vi) Carry out in vitro translation of the amplified mRNA to produce the protein encoded by the mRNA molecules at each spot in the microarray. There are several cell-free translation systems which can be employed to accomplish this step (U.S. Pat. No. 4,668,624 to Roberts (1987), and U S. Pat. No 5,556,769 to Wu, et al. (1996)). The most frequently used in vitro translation system consists of extracts from rabbit reticulocytes, which provides high efficiency of translation for eukaryotic RNA (either natural or in vitro generated). The proteins can be either in solution compartments or immobilized to a surface. Affinity labels/tags can be added during the unbiased amplification of full-length mRNA process or during in vitro translation to facilitate identification and/or isolation of the expression products. Among the useful specific labels/tags are fluorescence labels for detection or identification, histidine or biotin tags for isolation, and stable isotope labels for mass spectrometric identification Optionally, each protein in the matrix can be further characterized by mass spectrometric analysis. The end result of this procedure is an array of individual proteins, each occupying a spot in the array defined by the location of its specific capture tag.

The array of individual proteins thus produced has a number of embodiments.

(1) One of these embodiments is to allow rapid profiling of the proteins in a biological sample. This embodiment will find utility in understanding the expression pattern and cellular localization of a multitude of proteins simultaneously Both the spatial and temporal expression profiles can be readily followed due to the convenient format provided by the present invention Organ specific expression library (spatial expression profile) or expression library at specific development stage (temporal expression profile) can both be prepared to facilitate studies on biological interactions This application allows one to follow changes of not only one or few proteins, but all proteins expressed in a given type of cells simultaneously during biological development, disease monitoring, therapy, or learning process

(2) The second embodiment of the array of individual proteins is to analyze natural interactions, among which are biologically significant protein-protein interactions, protein (enzyme)-substrate interactions involved in normal biological activities in living cells. Proteins or peptides can be evaluated for binding to individual proteins in a protein array to examine their interaction targets

(3) The third embodiment of the array of individual proteins is to provide a means for identifying the protein targets for small molecules that are of pharmaceutical importance Small-molecule drug candidates can be evaluated for binding to individual proteins in a protein array to find targets for drugs, locate the likely causes of side effects of drugs, and engineer around the problems

(4) In the fourth embodiment of the array of individual proteins, proteins in a biological sample can be expressed, and individual proteins of interest can be further characterized to identify genetic variation. The human genome project revealed that the human genome has about 1.5 million SNPs (single nucleotide polymorphisms)—reflecting human variation. Since subtle structural changes could significantly alter protein function, binding assays using protein arrays prepared from organ-specific expression would provide a direct measure of the consequences of SNPs. As an example, cocaine acts on the re-uptake transporters for dopamine and other monoamine neurotransmitters. By constructing organ-specific (brain in this case) expression arrays, the activities of each subtype of dopamine transporters can be evaluated through binding assays with cocaine Such studies could shed light on our understanding of addiction-related brain changes, and why some people are more vulnerable than other to substance abuse

(5) Finally, the embodiment of the array of individual proteins is to profile a biological activity spatially (in different organs or individuals) and temporally (in different development stage) is another key technology of the present invention Unlike other profiling techniques which are based only on structural differences, the present invention can profile the biological activities of the whole spectrum of proteins expressed in a biological system, as measured through binding assays or enzymatic activity assays. Furthermore, the biological activity profiling can be carried out either on individual proteins in an expression array or mixtures of proteins by pooling individual proteins so as to include (or enhance) or exclude (or decrease) a protein Such functional profiling provides a means of evaluating the function of a given biological process

What is claimed is:

1. The method of making an in vitro amplification of heterogeneous full length mRNA comprising the following steps:
(a) isolating mRNA from biological samples;
(b) removing the 5′-phosphates from truncated mRNAs and non-mRNAs with calf intestinal phosphatase (CIP), which leaves the capped mRNAs unaffected;
(c) removing the 5′ end cap structure (Gppp.triphosphate) from the full-length mRNAs, leaving a 5′-monophosphate for subsequent ligation;
(d) adding a synthetic polynucleotides adapter containing an RNA polymerase promoter sequence (such as T7) to 5′ end of the decapped mRNAs;
(e) synthesizing first-strand cDNAs with reverse transcriptase and an anchor oligo-dT;
(f) synthesizing double-strand cDNAs using DNA polymerase (such as Pfu DNA polymerase) and a capturable DNA oligonucleotide primer complementary to the RNA adapter;
(g) capturing full-length cDNAs on a solid phase through specific binding interaction between the first moiety (e.g. biotin) at the 5′ terminus of the primer and the second moiety (e.g. streptavidin) bound to the solid support;
(h) using the captured full-length cDNAs for in vitro transcription to produce mRNAs.
(i) repeating the steps (a) through (h), if necessary, in order to obtain a large amount of amplified mRNA.

2. The method as defined in claim 1, wherein said synthetic polynucleotide adapter refers to RNA and DNA and as well as nucleotide analogs.

3. The method as defined in claim 2, wherein said nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like.

4. The method as defined in claim 1, wherein said RNA polymerase promoter is T3 RNA polymerase promoter.

5. The method as defined in claim 1, wherein said RNA polymerase promoter is T7 RNA polymerase promoter.

6. The method as defined in claim 1, wherein said RNA polymerase promoter is SP6 RNA polymerase promoter.

7. The method as defined in claim 1, wherein said RNA polymerase promoter is M13 RNA polymerase promoter.

8. The method according to claim 1, step (i) further comprising the steps of preparing probes for microarray hybridization, and for cDNA library construction, gene cloning, and the like.

9. The method according to claim 1, step (i) further comprising the steps of preparing mRNA/cDNA-based expression arrays.

10. The method according to claim 1, step (i) further comprising the steps of incorporating specific moieties/tags into the transcription products to facilitate the identification, characterization, or profiling of the said products.

11. The method according to claim 1, step (i) further comprising the steps of in vitro translation of the amplified transcription products and incorporating specific moieties/tags into the translation products to facilitate the identification, characterization, or profiling of the said products.

12. The method according to claim 11, wherein said the moieties/tags comprises a binding domain which is derived from a polypeptide selected from the group consisting of glutathione-S-transferase (GST), maltose-binding protein, chitin, cellulase, thioredoxin, avidin, streptavidin, green-fluorescent protein (GFP), Protein L and Protein G/A.
